(19) United States
(12) Patent Application Publication
(10) Pub. No.: US 2007/0000489 A1
(43) Pub. Date: Jan. 4, 2007

(54) Multi-function respiratory device

(76) Inventor: Shao-shih Huang, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/430,003

(22) Filed: May 9, 2006

Related U.S. Application Data
(63) Continuation-in-part of application No. 11/169,626, filed on Jun. 30, 2005, now abandoned.

(51) Int. Cl. A61M 16/00

(52) U.S. Cl. 128/204.18

(57) ABSTRACT

A multi-function respiratory device includes a control unit, a humidifier, and an air supply unit, which is reconfigurable to alternatively provide humidified and non-humidified air. Both the control unit and the humidifier comprise a trough and an air passage at one side. The humidifier further comprises a strip and an air passage at another side. The air supply unit comprises a strip and an air passage at one side. The troughs comprise lugs inwardly corresponding to the strips. Each air passage corresponds to the others. Thus, the control unit, the humidifier, and the air supply unit are combined together to provide humidified air; the control unit and the air supply unit are combined together to provide regular air; the control unit and the humidifier are combined together to form a single humidifying device.

This application is a continuation in part of my application filed Jun. 30, 2005, Ser. No. 11/169,626.

### BACKGROUND OF THE INVENTION

1. Field of the Invention

This invention relates to a respiratory device, in particular to a combination of a multi-function respiratory device reconfigurable to alternatively provide humidified air and regular air to a patient in hospital or at home.

2. Description of the Related Prior Art

A respiratory device is a standard apparatus in hospital. In most emergency rooms, the air supply system is embedded in the wall so that a user is simply inserted with a pipe to receive the air supply, and some patients use it at home.

Some respiratory apparatuses are combined with an air supply unit and a control unit, or even with a humidifier, thus when the user requires humidified air, they may operate the machine to provide as they need. The humidifier is an extra device which increases the cost, other than that, the weight and size are also increased.

There are some respiratory devices as taught in U.S. Pat. Nos. 5,673,687 and 6,554,260. However, these devices are designed to provide only humidified air supply, not to provide with regular air supply.

### SUMMARY OF THE INVENTION

It is the primary object of the present invention to provide a multi-function respiratory device, which is reconfigurable to alternatively provide regular air and humidified air.

It is another object of the present invention to provide a multi-function respiratory device, which is compact in size and is easy to carry and to store.

The present invention includes a control unit, a humidifier, and an air supply unit. The humidifier and the air supply unit are modularly disposed for selective operable coupling to the control unit one independent of the other. Both the control unit and the humidifier comprise a trough and an air passage at one side. The humidifier further comprises a strip and an air passage at another side. The air supply unit comprises a strip and an air passage at one side. The troughs comprise lugs inwardly corresponding to the strips. Each air passage corresponds to the others.

By a simple combination, the present invention is to provide a multi-function respiratory device, which is cost-effective.

### BRIEF DESCRIPTION OF THE DRAWINGS

FIG. 1 is an exploded view of the present invention;

FIG. 2 is a side view of the present invention;

FIG. 3 is a side view of a second embodiment of the present invention; and

FIG. 4 is a side view of a third embodiment of the present invention.

### DETAILED DESCRIPTION OF THE PREFERRED EMBODIMENTS

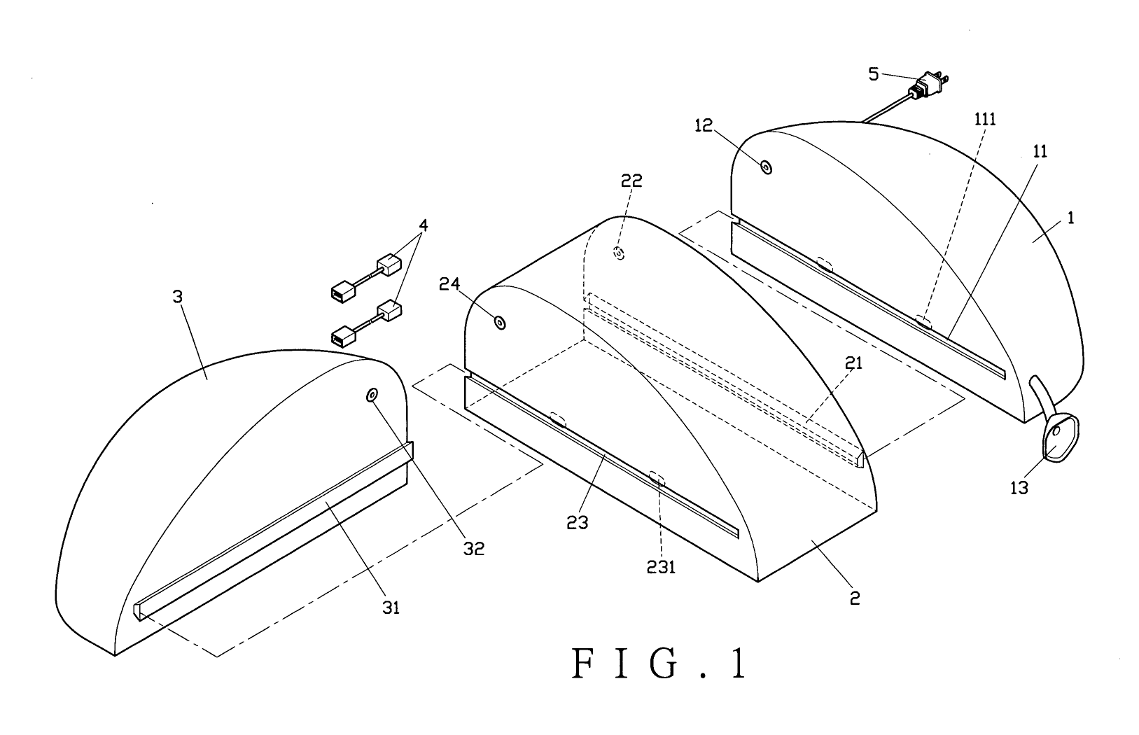

As shown in FIG. 1, the present invention comprises a control unit 1, a humidifier 2 and an air supply unit 3.

The control unit 1 has a trough 11 and an air passage 12 at one side. The trough 11 comprises lugs 111 inwardly. The control unit 1 is provided with an air tube 13 to supply air there through to a user.

The humidifier 2 comprises a strip 21 and an air passage 22 at one side corresponding in position to the trough 11 and the air passage 12 of the control unit 1, respectively. The humidifier 2 further comprises a trough 23 and an air passage 24 at the other side. The trough 23 comprises lugs 231 inwardly.

The air supply unit 3 may be a blower, a compressor or even an air supply system direct installed into the building of a hospital. The air supply unit 3 comprises a strip 31 and an air passage 32 at one side corresponding in position to the trough 11 and the air passage 12 of the control unit 1, or corresponding in position to the trough 23 and the air passage 24 of the humidifier 2.

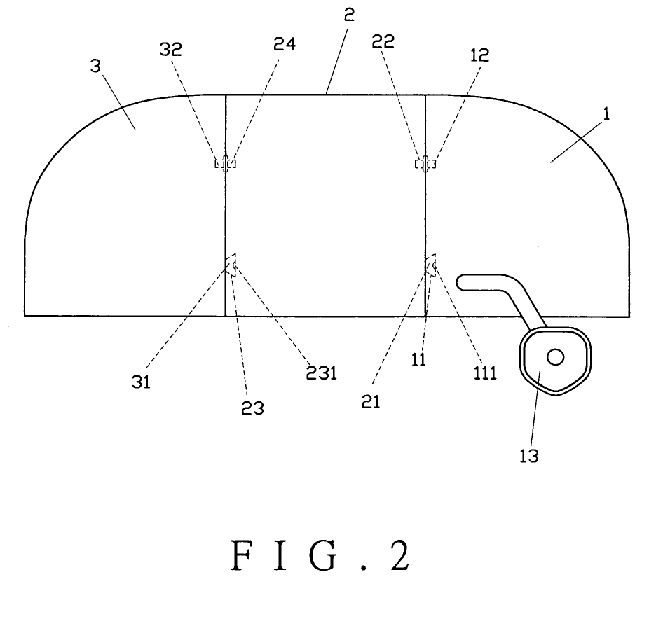

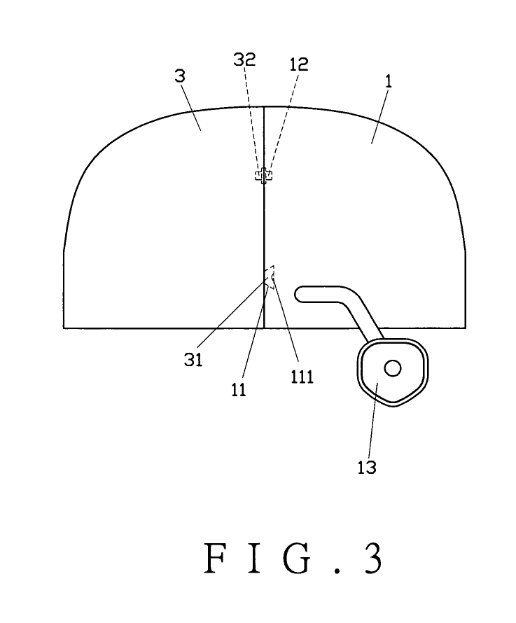

The control unit 1, the humidifier 2 and the air supply unit 3 comprise signal lines 4 and a power cord 5 at their respective backs, which are not to be claimed, and therefore will not be discussed hereinafter. Furthermore, as shown in FIG. 2 and FIG. 3, the control unit 1, the humidifier 2 and the air supply unit 3 are reconfigurable to alternatively provide humidified and non-humidified air.

To practice, as shown in FIG. 2, the control unit 1, the humidifier 2 and the air supply unit 3 are mounted in sequence. The strip 21 of the humidifier 2 corresponds to the trough 11 of the control unit 1, and the air passage 22 of the humidifier 2 corresponds to the air passage 12 of the control unit 1. The lugs 111 in the trough 11 engage with the strip 21 to hold the control unit 1 and the humidifier 2 securely.

The strip 31 of the air supply unit 3 corresponds to the trough 23 of the humidifier 2, and the air passage 32 of the air supply unit 3 corresponds to the air passage 24 of the humidifier 2. The lugs 231 in the trough 23 engage with the strip 31 to hold the humidifier 2 and the air supply unit 3 securely. Thus, a respiratory device to provide humidified air is formed.

To operate the present invention, the air supply unit 3 outputs air through the air passages 32 and 24, the humidifier 2, the air passages 22 and 12 and the air tube 13 of the control unit 1 to the user.

FIG. 3 shows a second embodiment of the present invention, which combines the control unit 1 and the air supply unit 3. The strip 31 of the air supply unit 3 corresponds to the trough 11 of the control unit 1, and the air passage 32 of the air supply unit 3 corresponds to the air passage 12 of the control unit 1. The lugs 111 in the trough 11 engage with the strip 31 to hold the control unit 1 and the air supply unit 3 securely. Thus, a respiratory device to provide regular air is formed.

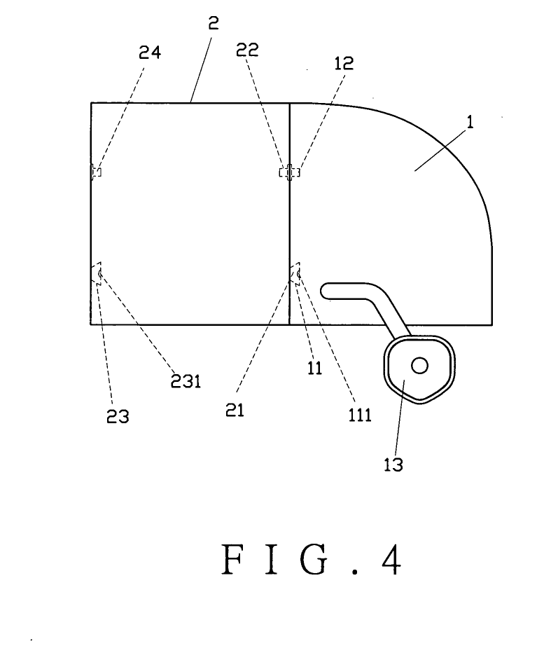

FIG. 4 shows a third embodiment of the present invention, which is to connect the control unit 1 and the humidifier 2 to an extra respiratory device to form a single humidifying device. The strip 21 of the humidifier 2 corresponds to the trough 11 of the control unit 1, and the air passage 22 of the humidifier 2 corresponds to the air passage 12 of the control unit 1. The lugs 111 in the trough 11 engage with the strip 21 to hold the control unit 1 and the humidifier 2 securely. Thus, a single humidifying device is formed. To operate, the extra respiratory device (not shown in the drawing) provides air through the air passage 24, the humidifier 2, the air passage 22 and the air tube 13 of the control unit 1 to the user.

I claim:

1. A multi-function respiratory device reconfigurable to alternatively provide humidified and non-humidified air comprising:
a control unit, and
a humidifier and an air supply unit modularly disposed for selective operable coupling to the control unit one independent of the other;
said control unit comprising a trough and an air passage at one side;
said humidifier comprising a strip and an air passage at one side corresponding in position to said trough and said air passage of said control unit, respectively, said humidifier further comprising a trough and an air passage at another side;
said air supply unit comprising a strip and an air passage at one side corresponding in position to either said trough and said air passage of said control unit or said trough and said air passage of said humidifier;
whereby said control unit, said humidifier and said air supply unit are combinable in sequence to provide humidified air; said control unit and said air supply unit being combined together in sequence to provide regular air.

2. The multi-function respiratory device, as recited in claim 1, wherein said troughs of said control unit and said humidifier comprise lugs inwardly.

3. The multi-function respiratory device, as recited in claim 1, wherein said air supply unit is a blower.

4. The multi-function respiratory device, as recited in claim 1, wherein said air supply unit is a compressor.

5. The multi-function respiratory device, as recited in claim 1, wherein said air supply unit is an air supply system installed in the building of a hospital.
